(19) United States
(12) Patent Application Publication
(10) Pub. No.: US 2016/0015647 A1
(43) Pub. Date: Jan. 21, 2016

(54) BISMUTH LIQUID FILLED HARD CAPSULES

(71) Applicant: CAPSUGEL BELGIUM NV, Bornem (BE)

(72) Inventors: Eric Ehrnsperger, New City, NY (US); Christopher Diorio, Campbell Hall, NY (US); Joseph J. Gross, Port Jervis, NY (US); John Lokhnauth, Fair Lawn, NJ (US)

(73) Assignee: CAPSUGEL BELGIUM NV, Bornem (BE)

(21) Appl. No.: 14/771,174

(22) PCT Filed: Mar. 3, 2014
(86) PCT No.: PCT/IB2014/000461

§ 371 Date: Aug. 27, 2015

Related U.S. Application Data
(60) Provisional application No. 61/774,173, filed on Mar. 7, 2013.

(51) Int. Cl. A61K 9/48; A61K 31/60

(52) U.S. Cl. CPC A61K 9/48; A61K 31/60; A61K 9/4825

(57) ABSTRACT

The present disclosure relates to a new, stable, oil based liquid suspension filled hard capsule dosage form for bismuth salts such as bismuth subsalicylate, a process for its manufacture, methods to deliver bismuth salts to humans or animals via stable hard capsules, and improved methods of treating gastrointestinal disorders with such dosage forms.

The present disclosure relates to a new, stable, oil based liquid suspension filled hard capsule dosage form for bismuth salts such as bismuth subsalicylate, a process for its manufacture, methods to deliver bismuth salts to humans or animals via hard capsules, and improved methods of treating gastrointestinal disorders with such dosage forms.

### BACKGROUND

Capsules are widely used in the pharmaceutical field as oral dosage forms for administration to humans and animals of, e.g., various active ingredients, including pharmaceuticals, veterinary products, and food and dietary supplements. Advantages of capsules over other conventional forms may include better patient compliance, greater flexibility in dosage form design, and less expensive manufacturing process.

Capsules are well-known dosage forms that normally consist of a shell filled with one or more specific substances. The shell itself may be a soft or a hard stable shell. Hard capsule shells are generally manufactured using dip molding processes, which can be distinguished into two alternative procedures. In the first procedure, capsules are prepared by dipping stainless-steel mold pins into a solution of polymer, optionally containing one or more gelling agents (e.g. carrageenans) and co-gelling agents (e.g. inorganic cations). The mold pins are subsequently removed, inverted, and dried to form a film on the surface. The dried capsule films are then removed from the molds, cut to the desired length, and then the telescoping fit caps and bodies are assembled, printed, and packaged. See, e.g., U.S. Pat. No. 5,264,223, U.S. Pat. No. 5,756,123, and U.S. Pat. No. 5,756,123. In the second procedure, no gelling agents or co-gelling agents are used and film-forming polymer solution gelifications on the molding pins are thermally induced by dipping pre-heated molding pins into the polymer solution. This second process is commonly referred to as thermogellation or thermogelling dip molding. See, e.g., EP 0401832, U.S. Pat. No. 3,493,407, U.S. Pat. No. 4,001,211, GB1310697, U.S. Pat. No. 3,617,588 and WO 2008/050209. The aforementioned manufacturing processes involve the use of solutions of the different ingredients that are needed for the making the telescoping fit hard capsule shells.

Hard capsules may be filled with active ingredients in manners known in the art. Typically active ingredients are combined with various compatible excipients for ease of fill. The resulting fill may be a dry powder, a granulation, a suspension, or a liquid. Suspension and liquid fills have various advantages over other fills, such as faster release profiles, faster delivery, and faster dispersion of insoluble and/or slightly soluble active ingredients. In particular, hard capsules filled with oily liquids may require sealing of the telescoping capsule parts.

Additionally, stable, filled hard capsules have advantages over other dosage delivery forms such as liquids and solid tablets. While patient compliance may be better (due to ease of dosing and administration) with oral dosage forms such as tablets and caplets when compared to liquids, certain active ingredients may be difficult to formulate into dry granules or may be otherwise incompatible with the tableting process. Further, additional processing steps (such as granulation and/or tableting) add complexity and resultant cost to pharmaceutical dosage forms. Of interest is the development of unitary pharmaceutical dosage forms which retain the advantages of purely liquid dosage forms without the difficulties of administration and patient compliance which occur with consumer-dosed liquid dosage forms. Another consideration is improved patient compliance for taste-masking, i.e., capsules being preferred by consumers over chewable tablets. Finally, bismuth salts are known for unpleasant taste and side effects in the mouth when administered in liquid or chewable formulations, such as the known side effects of blackened tongue when bismuth active ingredients come in contact with the mucosal membranes in the mouth. See, e.g., Worldwide Efficacy of Bismuth Subsalicylate in the Treatment of Travelers' Diarrhea, R. Steffens, Clin Infect Dis. (1990) 12 (Supplement 1): S80-S86.

Bismuth salts are known for the treatment of gastrointestinal disorders. The use of bismuth subsalicylate for antidiarrheal compositions has been disclosed, for example in U.S. Pat. No. 4,588,589 incorporated in its entirety herein. Aqueous liquid and tablet bismuth subsalicylate-containing compositions are also commercially available, for example, sold under the trademark PEPTO-BISMOL® (Procter & Gamble). Bismuth salts are nearly insoluble in aqueous solution. Typical pharmaceutically effective amounts of bismuth salts for administration to humans range from about 87 mg to about 524 mg (pediatric to adult) administered up to 4 times a day as needed. Lower doses may be administered more frequently, for example is about 87 mg may be administered every 30 min or 1 hour.

### DETAILED DESCRIPTION

One embodiment of the present disclosure relates to a liquid-filled pharmaceutical dosage form, comprising at least one bismuth salt and at least one oil in a hard capsule, and optionally at least one colorant, at least one co-solvent, at least one surfactant, or mixtures thereof, wherein the hard capsule is optionally sealed. Certain embodiments comprise fish oil, bismuth subsalicylate, and at least one colorant in a sealed hard gelatin capsule.

Certain embodiments comprise pharmaceutical dosage forms comprising a pharmaceutically effective amount of bismuth subsalicylate, and at least one oil selected from coconut oil distillation fraction of 55% triglycerides of C₈ and 45% triglycerides of C₁₀ fatty acids (MIGLYOL® 812), fish oil, coconut oil, and mixtures thereof, in a gelatin or HPMC hard capsule, and optionally at least one colorant, at least one co-solvent, at least one surfactant, or mixtures thereof, wherein the hard capsule is sealed. Certain pharmaceutical dosage form embodiments consist of a pharmaceutically effective amount of bismuth subsalicylate, and at least one oil selected from coconut oil distillation fraction of 55% triglycerides of C₈ and 45% triglycerides of C₁₀ fatty acids (MIGLYOL® 812), fish oil, coconut oil, and mixtures thereof, in a gelatin or HPMC hard capsule, and optionally at least one colorant, at least one co-solvent, at least one surfactant, or mixtures thereof, wherein the hard capsule is sealed.

In certain embodiments, the pharmaceutical dosage forms according to the present disclosure provide an effective amount of at least one bismuth salt for treatment of gastrointestinal disorders, and are suitable for administering to a mammal, preferably a human. In some embodiments according to the present disclosure, the treatment of gastrointestinal disorders is improved by the embodiments of the pharmaceutical dosage forms. These improvements may arise from more rapid dissolution, more effective application of the bismuth salts to the gastrointestinal tract, improved patient compliance, decreased side effects, and/or other advantages of the combination of the oil with the bismuth salt.

“Administering” refers to any method which delivers the dosage forms used in this disclosure to the subject in need thereof in such a manner so as to be effective in the treatment of the gastrointestinal disorder. Oral administration of the dosage forms is of particular interest.

Non-limiting examples of bismuth salts suitable for the present disclosure include, bismuth aluminate, bismuth subcarbonate, bismuth subcitrate, bismuth citrate, tripotassium dicitrato bismuthate, bismuth subgalate, bismuth subnitrate, bismuth tartrate, bismuth subsalicylate, and mixtures thereof. Certain embodiments include bismuth citrate, bismuth subsalicylate, and mixtures thereof. In one embodiment, the bismuth salt is bismuth subsalicylate (C₇H₅BiO₄).

In certain embodiments of the present disclosure, the pharmaceutical dosage forms further comprise food safe or pharmaceutical grade colorant compatible with an oil-based fill.

Co-solvents are optionally added to the oil or oils for use in certain embodiments. Suitable co-solvents include any solvent that is used to increase solubility of the at least one bismuth salt in the formulation in order to allow delivery of the desired dose per dosing unit or to enhance the miscibility or suspension behavior of the various formulation components. Suitable solvents include triacetin (1,2,3-propanetriyl triacetate or glyceryl triacetate available from Eastman Chemical Corp.) or other polyol esters of fatty acids, trialkyl citrate esters, propylene carbonate, dimethylisosorbide, ethyl lactate, N-methylpyrrolidones, diethylene glycol monoethyl ether (TRANSCUTOL® by Gattefosse), peppermint oil, 1,2-propylene glycol (PG), ethanol, oleic acid, and polyethylene glycols. In certain embodiment the solvents comprise triacetin, propylene carbonate (Huntsman Corp.), TRANSCUTOL® (Gattefosse), ethyl lactate (Purac, Lincolnshire, Nebr.), propylene glycol, oleic acid, dimethylisosorbide (sold under the trademark ARLASOLVE DMI®, ICI Americas), stearyl alcohol, cetyl alcohol, cetostearyl alcohol, glyceryl behenate, and glyceryl palmitostearate. The optional co-solvent may be a single co-solvent or mixtures of any co-solvents.

Hard capsules for use in certain embodiments include any telescoping, two piece capsule, including but not limited to a gelatin capsule, a pullulan capsule, and a hydroxypropyl methyl cellulose (HPMC) capsule. Certain embodiments of hard capsules include CONI-SNAP® capsules, DRCAPS™ capsules, OCEANCAPS™ fish gelatin capsules, PEARLCAPS® gelatin capsules, PLANTCAPS™ pullulan capsules, VCAPS® HPMC capsules, and VCAPS® PLUS HPMC capsules available from Capsugel. The capsules according to certain embodiments of the present disclosure, are sufficiently stable for administration to humans and other animals, and display good mechanical properties, i.e., no cracking, discoloring, sticking, and/or deformation. The capsules according to the present disclosure are non-leaking.

As used in the present disclosure, “gastrointestinal disorder,” relates to any infection, disease or other disorder of the gastrointestinal system, such as the upper and/or lower gastrointestinal tract. Such disorders include one or more of the following conditions: diarrhea, heartburn, indigestion, upset stomach, abdominal pain and/or cramping, flatulence, nausea, abdominal distention, fever, constipation, blood, mucus and/or pus present in feces, vomiting, gastroenteritis, weight loss, anorexia, malaise, and any other related condition.

Oils for use in certain embodiments are pharmaceutically acceptable or food grade oils suitable for administration to humans. Examples include but are not limited to digestible oils; including but not limited to fish oil, corn oil, vegetable oils such as soybean, safflower, corn, olive, cottonseed, arachis, sunflower seed, palm, and rapeseed oils, and mixtures thereof. In certain embodiments, the oil is selected from fish oil, olive oil, corn oil, soybean oil, coconut oil (commercially available as different distillation fractions, including, e.g., MIGLYOL® 812, 55% triglycerides of C₈ and 45% triglycerides of C₁₀ fatty acids), and mixtures thereof.

Surfactants for optional use in certain embodiments include, but are not limited to, non-ionic surfactants, or combinations of non-ionic surfactants and ionic surfactants. Non-limiting examples of suitable non-ionic surfactants include fatty acid esters, their amide or ether analogues, or hydrophilic derivatives thereof, such as: monoesters or diesters, or hydrophilic derivatives thereof, or mixtures thereof; monoglycerides or diglycerides, or hydrophilic derivatives thereof, or mixtures thereof; mixtures having enriched mono- or/and diglycerides, or hydrophilic derivatives thereof; monoesters or diesters or multiple-esters of other alcohols, polyols, saccharides or oligosaccharides or polysaccharides, oxyalkylene oligomers or polymers or block polymers, or hydrophilic derivatives thereof, or the amide analogues thereof; and fatty acid derivatives of amines, polyamines, polyimines, aminoalcohols, aminosugars, hydroxyalkylamines, hydroxypolyimines, peptides, polypeptides, or the ether analogues thereof. Surfactants comprising, or enriched in, fatty acid moieties having 6 to 12 carbon atoms. In certain embodiments, the fatty acid moieties have 6 to 8, 6 to 10, 6 to 12, 8 to 10 or 8 to 12 carbon atoms. The term “hydrophilic derivatives” as used herein means surfactants derivatized with hydrophilic components such that additional hydrophilic moieties are added to the surfactant molecules or to a partial structure of the surfactant molecules. Hydrophilic derivatives of surfactants also include partially derivatized surfactants, which are a mixture of the surfactant and its hydrophilic derivatives. As such, products of transesterification or other similar transformations of oils, alcohols and other surfactants with hydrophilic materials such as PEG, polypropylene glycol, saccharides, oligosaccharides, polysaccharides, and polyols, are included in certain embodiments.

Another class from which surfactants may be chosen for certain embodiments is the ionic or Zwitterionic surfactants, such as fatty acid salts, bile salts, sulfates, sulfonates, sulfosuccinates, carboxylates, lactylates, phospholipids and derivatives, quaternary ammonium salts, amine salts, polyethoxylated ammonium salts, and mixtures thereof. Hydrophilic derivatives of such surfactants, such as PEG-phospholipids, are also included.

Additional components or excipients may optionally be added, such as other active ingredients and/or other excipients, for example, magnesium aluminum silicate, or other suspension aiding adjuvants.

Certain embodiments of the pharmaceutical dosage forms may advantageously be sealed, by administration of a sealing solution applied to the hard capsules by hand, or by automatic or mechanical means such as LEMS® 70 System liquid encapsulation microspray sealing, and CFS technology (CFS 1200 liquid capsule filling and sealing system and CFS 1500C containment capsule filling and sealing system) available from Capsugel. See, e.g., U.S. Pat. No. 7,645,407; EP 2083787.

The following examples are merely illustrative, and should not be construed as limiting the present disclosure

### Example 1

Twelve grams of MIGLYOL® 812 and 15 mg of FD&C Red 27 were added to a beaker and mixed, using a stir bar on a stir plate (for larger batches an overhead mixer was used). Six grams of bismuth subsalicylate were weighed out and add slowly while mixing. After all of the bismuth subsalicylate was suspended, the resulting suspension was allowed to mix for an additional 5 minutes. For small prototyping, the suspension was weighed into a size 00 hard gelatin capsule (alternatively, HPMC capsules could be used). The weight of suspension per capsule was 785 mg, which is equivalent to 262 mg of bismuth subsalicylate per capsule. The capsules were sealed manually using a 1:1 ethanol/water sealing solution placed with a spatula or glass rod at the interface of the cap and body (approximately 50 μl) at room temperature.

Multiple capsules were made in this manner, put into scintillation vials and placed into various commercially available stability chambers. The capsules were stored at 40° C./75% relative humidity (“RH”) and at 25° C./ambient humidity. The capsules were observed for physical appearance at 1 month, 3 months, and 6 months for 40° C./75% RH conditions. The capsules were observed for physical appearance at 3 months, 6 months, and 9 months at 25° C. ambient humidity conditions. Good stability without color change, separation, or leakage was observed. The results are shown in Table 1.

The capsules were tested using Apparatus 2 with 900 mL of 0.1 N HCl at 37 C., 50 RPM with paddles. The capsules were weighed down using stainless steel spiral sinkers. The hard gelatin capsules started to release the suspension at 1 min and the capsule was fully open at 3 minutes.

TABLE 1

| 1 month at | 3 months at | 6 months at |
|---|---|---|
| 40° C./75% RH | 40° C./75% RH | 40° C./75% RH |
| No separation or | No separation or | Slight separation of |
| color change. | color change. | suspension. Mild shaking |
| No leakage. | No leakage. | of capsule reconstitutes. |
| | | No leakage. |
| 3 months | 6 months | 9 months |
| at 25° C. | at 25° C. | at 25° C. |
| No separation or | No separation or | No separation or |
| color change. | color change. | color change. |
| No leakage. | No leakage. | No leakage. |

### Example 2

Twelve grams of MIGLYOL® 812, 3.18 grams of phosphatidyl choline (Phosal 75 SA), and 15 mg of FD&C Red 27 were added to a beaker and mixed using a stir bar on a stir plate (for larger batches an overhead mixer was used). Six grams of bismuth subsalicylate was weighed out and added slowly while mixing. After all of the bismuth subsalicylate was suspended, the suspension was allowed to mix for an additional 5 minutes. For small prototyping, the suspension was weighed into a size 00 hard gelatin capsule (alternatively, HPMC capsules could be used). The weight of suspension per capsule was 926 mg which is equivalent to 262 mg of bismuth subsalicylate per capsule. The capsules were sealed manually using a 1:1 ethanol/water sealing solution placed with a spatula or glass rod at the interface of the cap and body (approximately 50 μl) at room temperature.

The capsules were tested using Apparatus 2 with 900 mL of 0.1 N HCl at 37C, 50 RPM with paddles. The capsules were weighed down using stainless steel spiral sinkers. The hard gelatin capsules started to release the suspension at 1 minute and the capsule was fully open at 3 minutes. The formulation was dispersed in the aqueous environment.

### Example 3

Twelve grams of MIGLYOL® 812, 2 grams of propylene glycol, and 15 mg of FD&C Red 27 were added to a beaker and mixed using a stir bar on a stir plate (for larger batches an overhead mixer was used). Six grams of bismuth subsalicylate was weighed out and added slowly while mixing. After all of the bismuth subsalicylate was suspended, the suspension was allowed to mix for 5 minutes. For small prototyping, the suspension was weighed into a size 00 hard gelatin capsule (alternatively, HPMC capsules could be used). The weight of suspension per capsule was 873 mg which is equivalent to 262 mg of bismuth subsalicylate per capsule. The capsules were sealed manually using a 1:1 ethanol/water sealing solution placed with a spatula or glass rod at the interface of the cap and body (approximately 50 μl) at room temperature.

### Example 4

Twelve grams of fish oil and 15 mg of FD&C Red 27 were added to a beaker and mixed using a stir bar on a stir plate (for larger batches an overhead mixer was used). Six grams of bismuth subsalicylate was added slowly while mixing. After all of the bismuth subsalicylate was suspended, the suspension was allowed to mix for an additional 5 minutes. For small prototyping, the suspension was weighed into a size 00 hard gelatin capsule (alternatively, HPMC capsules could be used). The weight of suspension per capsule was 785 mg which is equivalent to 262 mg of bismuth subsalicylate per capsule. The capsules were sealed manually using a 1:1 ethanol/water sealing solution placed with a spatula or glass rod at the interface of the cap and body (approximately 50 μl) at room temperature.

### Example 5

Six grams of bismuth subsalicylate were added with stirring to twelve grams of MIGLYOL® 812 and 15 mg of FD&C Red, along with 0.54 g of magnesium aluminum silicate as a suspension aid and placed into sealed capsules as described previously. Stability of the capsules and the fill was acceptable at room temperature for 6 months. Visual inspection showed stable suspension filled capsules.

Other embodiments of the disclosure will be apparent to those skilled in the art from consideration of the specification and practice of the disclosure herein. It is intended that the specification and examples be considered as exemplary only, with a true scope and spirit of the disclosure being indicated by the following claims.

1. A pharmaceutical dosage form comprising a pharmaceutically effective amount of at least one bismuth salt and at least one oil in a hard capsule.

2. The pharmaceutical dosage form according to claim 1, further comprising at least one of at least one colorant, at least one co-solvent, at least one surfactant, and mixtures thereof.

3. The pharmaceutical dosage form according to claim 1, wherein the hard capsule is optionally sealed.

4. The pharmaceutical dosage form according to claim 1, wherein the at least one oil is selected from coconut oil, fish oil, and mixtures and distillation sub-fractions thereof.

5. The pharmaceutical dosage of claim 1, wherein the at least one bismuth salt is selected from bismuth alum inate, bismuth subcarbonate, bismuth subcitrate, bismuth citrate, tripotassium dicitrato bismuthate, bismuth subgalate, bismuth subnitrate, bismuth tartrate, bismuth subsalicylate, and mixtures thereof.

6. The pharmaceutical dosage form of claim 1, wherein the at least one bismuth salt is bismuth subsalicylate.

7. The pharmaceutical dosage form of claim 1, wherein the hard capsule is selected from a gelatin capsule, a pullulan capsule, and a hydroxypropyl methyl cellulose (HPMC) capsule.

8. The pharmaceutical dosage form of claim 1, wherein the pharmaceutical dosage form is stable for at least one month.

9. The pharmaceutical dosage form of claim 1, wherein the pharmaceutical dosage form is stable for at least six months.

10. (canceled)

11. A pharmaceutical dosage form comprising:
a pharmaceutically effective amount of bismuth subsalicylate, and at least one oil selected from coconut oil distillation fraction of 55% triglycerides of C₈ and 45% triglycerides of C₁₀ fatty acids (MIGLYOL® 812), fish oil, coconut oil, and mixtures thereof, in a gelatin or HPMC hard capsule, and optionally at least one colorant, at least one co-solvent, at least one surfactant, or mixtures thereof, wherein the hard capsule is sealed.

12. A method of improved treatment of a gastrointestinal disorder in a human or other animal in need thereof, comprising administration of a pharmaceutical dosage form of claim 1.

13. A method of manufacturing a pharmaceutical dosage form according to claim 1.

14. A pharmaceutical dosage form consisting essentially of:
bismuth subsalicylate and at least one oil selected from coconut oil distillation fraction of 55% triglycerides of C₈ and 45% triglycerides of C₁₀ fatty acids (MIGLYOL® 812), fish oil, coconut oil, and mixtures thereof, in a gelatin or HPMC hard capsule.

15. A pharmaceutical dosage form consisting essentially of:
bismuth subsalicylate;
at least one oil selected from coconut oil distillation fraction of 55% triglycerides of C₈ and 45% triglycerides of C₁₀ fatty acids (MIGLYOL® 812), fish oil, coconut oil, and mixtures thereof, in a gelatin or HPMC hard capsule;
one or more colorants;
one or more co-solvents;
one or more surfactant; and
wherein the hard capsule is sealed.
